(19) United States
(12) Patent Application Publication
(10) Pub. No.: US 2005/0256195 A1
(43) Pub. Date: Nov. 17, 2005

(54) Composition for preventing hypertension

(76) Inventors: Kenji Tayama, Aichi (JP); Shino Kondou, Aichi (JP); Sumio Akita, Aichi (JP); Yoshinori Tsukamoto, Aichi (JP)

Correspondence Address: BIRCH STEWART KOLASCH & BIRCH, PO BOX 747, FALLS CHURCH, VA, 22040-0747, US

(21) Appl. No.: 10/506,325

(22) PCT Filed: Mar. 4, 2002
(86) PCT No.: PCT/JP02/01967

§ 371 Date: Sep. 2, 2004

(51) Int. Cl. A61K 31/19

(52) U.S. Cl. 514/557

(57) ABSTRACT

This invention relates to a composition that can inhibit the elevation of blood pressure, does not generate any side effects, and can be safely used. Particularly, this invention relates to food or a pharmaceutical composition for preventing hypertension that can inhibit the elevation of blood pressure via the long-term oral ingestion thereof and comprises at least one member selected from among acetic acid, acetate ion, and acetate.

### TECHNICAL FIELD

The present invention relates to a composition for preventing hypertension. Particularly, the present invention relates to food or a pharmaceutical composition that has the effect of inhibiting the elevation of blood pressure via the long-term oral ingestion thereof and thus can be used for preventing hypertension.

### BACKGROUND ART

Recently, the need for prevention of lifestyle-related diseases has been actively argued. However, the number of patients with arteriosclerosis, that of a variety of lifestyle-related diseases recognized as signal symptoms thereof, and that of patients-to-be thereof have not yet decreased. Thus, analyses have been undertaken via a wide variety of approaches in order to treat lifestyle-related diseases, and the development of pharmaceutical agents has been extensively advanced.

Hypertension is a symptom of lifestyle-related diseases that has a large number of patients or patients-to-be. According to the Japan's national nutrition survey in 1998 (Kokumin eiyou no genjo (Current Status of National Nutrition), Kenko Eiyo Joho Kenkyukai (Society for Health and Nutrition Studies) (ed.), Dai-ichi Shuppan Publishing, Co., Ltd., p. 54, 2000), as many as 25.3% of males and 20.6% of females were evaluated as being afflicted with hypertension, and 19.8% of males and 14.5% of females were evaluated as being afflicted with borderline hypertension, based on the blood pressure levels of surveyed males and females aged 15 or older. The results of this survey indicate that ten million or more people are hypertension patients-to-be in Japan. This is a seriously problematic situation. Although the cause for hypertension has not yet been clarified, interactions between a predisposing cause (genetic) and the environment (lifestyle) are considered to be causative of hypertension.

Development of an agent for ameliorating hypertension, i.e., an antihypertensive agent, has been remarkable, and diuretics, sympatholytic agents (α1 blockers or β blockers), ACE inhibitors, calcium antagonists, and angiotensin II receptor antagonists have been used. These agents, however, generate side effects and thus must be used by being prescribed by a specialist with careful and strict control. Real effects of antihypertensive agents cannot be attained via ingestion thereof only when a patient does not feel well. Antihypertensive agents become effective via the long-term ingestion in adequate amounts. Antihypertensive agents are considered to be effective for prevention. Because they are pharmaceutical agents, however, long-term ingestion thereof for a preventive purpose imparts a serious economic burden, such as increased medical expenses, on patients. Accordingly, discovery of an inhibitor for the elevation of blood pressure, which can be casually used by anybody, is more cost-effective, generates no substantial side effects, and can be easily obtained, has been desired.

### DISCLOSURE OF THE INVENTION

The objects of the present invention are to discover a substance that inhibits the elevation of blood pressure via the long-term oral ingestion thereof, generates no substantial side effects, is very cost-effective, and is easily obtainable, and to provide food or a pharmaceutical composition comprising such substance.

In order to attain the above objects, the present inventors have conducted studies as described below.

(1) Examination of Screening System

In order to search for a substance that inhibits the elevation of blood pressure from among a variety of substances that can be casually used by anybody, are inexpensive, generate no substantial side effects, and can be easily obtained, in vitro screening that focuses on responses by cultured cells, organs, or enzymes is usually carried out. A substance that was found positive via in vitro screening is not always found positive via an in vivo experiment, i.e., an animal experiment. In contrast, a substance that is effective in vivo may not exhibit its effect via in vitro screening that is slightly different from the actual conditions in organisms. Therefore, the present inventors were persistent in implementing cost- and time-consuming animal experiments. In the animal experiments, they raised test animals for a long time period of 2 months and conducted screening of a large number of highly safe substances.

Specifically, samples prepared by adding a variety of purified substances to the standard feeds to a final concentration of 0.3% and then mixing them were administered to SHR rats, i.e., the animal models of essential hypertension, that gradually develop hypertension as they grow older week-by-week. The effects of sample administration were evaluated by using 1 SHR (spontaneouly hypertensive rat) per sample, letting it live for 8 weeks, and comparing its blood pressure level with the mean of the control group (6 individuals) to which the purified substance had not been administered. In general pharmacological tests, animals, such as rats, are forced to orally ingest an aqueous solution of pure reagent every day in predetermined amounts relative to their body weights. The present inventors considered that a system in which an active substance is administered at substantially the same time as a meal was suitable in terms of higher safety. Thus, they adopted a method in which a test substance was mixed with feed. As a result of the experiment via this screening system, the present inventors found that pure acetic acid added to feed has the effect of alleviating the elevation of blood pressure in SHR. They confirmed this finding via another experimentation utilizing an increased number of rats and simultaneously found that the increased dose resulted in improved effects of inhibiting the elevation of blood pressure.

(2) Correlation Between Hypertension and Acetic Acid

Concerning the correlation between hypertension and acetic acid, Jinko Zoki (Artificial Organ), (vol. 21, p. 958, 1992) describes that patients who have been receiving dialysis for a long period of time are likely to have recurring hypertension with high frequency after acetic acid dialysate containing acetic acid of high concentration (35 mM) is changed to bicarbonate dialysate containing acetic acid of medium concentration (8 mM).

(3) Correlation Between Acetic Acid Ingestion and Acetic Acid Concentration in Blood

Orally ingested acetic acid is known to be rapidly changed into acetyl-CoA in the body and then metabolized. Also, most thereof is completely degraded to carbon dioxide and water. Despite the aforementioned finding, accordingly, it is impossible to maintain acetic acid concentration in blood at a constantly high level via oral ingestion of an acetic-acid-containing composition alone.

As described in the Reference Example below, the present inventors verified the difficulty of maintaining the acetic acid concentration in blood via oral ingestion of an aqueous solution of acetic acid in an experiment using pigs. Specifically, pigs were forced to orally ingest an aqueous solution of acetic acid. The pigs were dissected for analysis over time, and the acetic acid concentrations in the blood of various sites were then measured. In this case, pigs were forced to ingest a solution containing 6 g of acetic acid per 1,000 g of solution (an aqueous solution of acetic acid: about 100 mM) at one time. The maximal acetic acid concentration in blood was exhibited in the hepatic portal vein 10 minutes after ingestion, and the concentration at this site was 0.8 mM. The concentration was 0.4 mM in the abdominal artery, and it was as low as 0.18 mM in the postcaval vein. Further, the concentrations were lowered to 0.5 mM, 0.3 mM, and 0.17 mM in the hepatic portal vein, in the abdominal artery, and in the postcaval vein, respectively, 30 minutes after ingestion. The following was found based on the correlation with the dilution of acetic acid with body fluid or the rate of absorption. That is, orally ingested acetic acid migrates to the portal vein while being diluted 125-fold or more, the rate of acetic acid metabolism in the body is rapid, the acetic acid concentration in blood is rapidly lowered, and the acetic acid concentration in the vein does not substantially change.

Accordingly, it is difficult to maintain highly concentrated acetic acid in blood to an extent such that blood pressure is lowered in spite of ingestion of an acetic acid solution of ingestible concentration.

According to other literature (Masui to sosei (Narcosis and Anabiosis), vol. 26, p. 63, 1990), drip-feeding of 1 liter of acetated Ringer's solution at the time of surgical operation actually resulted in elevated acetic acid concentration in blood during the operation. However, blood pressure at that time was somewhat elevated instead of being lowered. Thus, the fact that blood pressure cannot be lowered with acetic acid of a physiological concentration has been verified, and the acetic acid concentration in blood was found via experiment to return to the level before the operation when the patient was awake after operation. The rate of acetic acid metabolism was, therefore, determined to be very rapid. In this literature, 15 males and females were subjected to measurement of the acetic acid concentrations in blood immediately before the initiation of test drug administration, and the measured values were 0 to 0.5 mg/dl according to the graph. When the surgical operation was completed, the acetic acid concentration was elevated to 0.5 to 2.5 mg/dl (mean: 1.5 mg/dl) as a result of drip-feeding of acetic acid, and the blood pressure level was somewhat elevated in comparison with that immediately before the initiation of test drug administration, although this elevation was not significant. The present inventors independently conducted measurement of a large number of acetic acid concentrations in human blood under fasting conditions. The maximal concentration was 0.6 mg/dl (0.1 mM) and the minimal concentration was 0 (undetected), which were consistent with the results attained in the aforementioned literature.

The following points were elucidated: (i) acetic acid does not lower blood pressure at a concentration in human blood of around 0.1 to 0.4 mM; (ii) the acetic acid concentration in human blood under fasting conditions is 0.1 mM or lower; and (iii) the acetic acid concentration in blood does not substantially change via oral ingestion of vinegar at the time thereof. Specifically, oral ingestion of vinegar cannot rapidly lower blood pressure.

(4) Examination of Report on the Effect of Vinegar

An example of a composition comprising highly concentrated acetic acid is vinegar as a condiment. It is described in health magazines or health-related books that ingestion of black vinegar, which is a kind of vinegar, lowers the blood pressure level of a hypertensive person. However, no active ingredient thereof is described at all. In the academic literature (Kiso to rinsho (Experimental and Clinical Medicine), vol. 19, p. 237, 1985), the active ingredient of black vinegar was searched for, although it has not yet been identified. Fractions having high activity of inhibiting angiotensin converting enzyme (ACE), which plays a role in blood pressure regulation, were subjected to amino acid analysis. As a result, a wide variety of amino acids had been detected. This suggests that the active ingredient of black vinegar is a peptide or amino acid.

A substance that inhibits ACE activity in vitro may also be able to lower blood pressure in vivo. Accordingly, the degree of the ACE activity inhibited is also inspected. Tsuzuki et al. inspected the substance in vinegar that inhibits ACE activity (Journal of the Japanese Society for Food Science and Technology, vol. 39, p. 188, 1992), and they concluded that an organic acid is not involved with ACE inhibition.

Matsui et al. demonstrated the effects of ginseng vinegar to inhibit the elevation of blood pressure in a stroke-prone spontaneously hypertensive rat (SHRSP) (Yakuri to chiryo (Pharmacology and Treatment), vol. 26, p. 23, 1998), and they stated in the “Consideration” section that ginseng vinegar may have an antihypertensive effect since the antihypertensive effect of the ginseng extract has already been reported.

Even though vinegar is known to have a antihypertensive effect from the aforementioned report, vinegar comprises only about 4% to 5% of acetic acid. Thus, it is appropriate to consider that such effect was exhibited by “a starting material or a functional component generated via processing thereof” instead of “the acetic acid.” Also, it is difficult to compare the effect attained by a single ingredient with that attained by food comprising a wide variety of ingredients such as vinegar. Among the wide variety of ingredients, some ingredients can positively act on a certain symptom while some other ingredients can adversely act thereon. Therefore, the effect of food is integrated effects of all the ingredients thereof.

Thus, the excellent antihypertensive effect attained by the long-term oral ingestion of a very small amount of pure acetic acid was discovered by the present inventors. The present invention has been completed based on such finding.

Acetic acid itself have an acidic taste. Thus, it is practically difficult to ingest acetic acid of high concentration. Even if it is well-diluted, it is difficult to ingest a large amount of acetic acid. Accordingly, ingestion of acetic acid within an adequate concentration range becomes necessary. In order to ameliorate the acidic taste, acetic acid can be neutralized with alkali, or a large amount of other taste components can be added to acetic acid. This is not simple, however, since it involves problems such as increased harsh unpleasant taste, overconsumption of minerals, or nutritional imbalances. In the present invention, therefore, the concentration that was adequate for ingestion was also examined, and it was determined that the preferable amount of acetic acid molecules to be included was 0.36 g to 30 g per 1,000 g of composition.

This value was converted to human terms. This demonstrates that ingestion of an average of 0.5 g to 5 g of acetic acid per day by an adult who weighs 60 kg delays hypertension. That is, such ingestion is effective for preventing hypertension.

Further, elevation of blood pressure can be inhibited after acetic acid has been continually ingested for 3 weeks. On the contrary, this effect cannot be expected to a significant extent by the short-term ingestion of within 2 weeks. Ingestion of acetic acid for a long time period of at least 3 weeks leads to an effect of inhibiting the elevation of blood pressure.

Continual ingestion of the composition according to the present invention was found to prevent a person who may develop hypertension in the future or a person whose blood pressure is at a borderline level (a systolic blood pressure of 140 to 180 mmHg) from developing hypertension, according to the results of the experiment.

The present invention has been completed based on the aforementioned findings. Specifically, the present invention includes the following inventions.

(1) Food or a pharmaceutical composition for preventing hypertension, which inhibits the elevation of blood pressure via the long-term oral ingestion thereof and comprises at least one member selected from among acetic acid, acetate ion, and acetate.

(2) The composition according to (1), which comprises at least one member selected from among acetic acid, acetate ion, and acetate in amounts of 0.36 g to 30 g in total (in terms of acetic acid) per 1,000 g of composition.

(3) The composition according to (1) or (2), wherein the intake of at least one member selected from among acetic acid, acetate ion, and acetate is adjusted to 0.5 g to 5 g in total (in terms of acetic acid) per day.

(4) The composition according to any of (1) to (3), wherein the period of ingestion is 3 weeks or longer.

Hereafter, the present invention is described in detail.

The process for producing acetic acid that is employed in the present invention is not particularly limited. It may be produced by synthesis or fermentation. When it is used as food, however, use of acetic acid produced by fermentation, i.e., vinegar (fermented vinegar), is preferable from the viewpoint of consumer's perception. Especially, brown rice vinegar, the sour taste of which is less likely to stand out, or cider vinegar, which has a refreshing flavour, is preferable. The use of a sweetener or flavoring agent can provide acetic acid having milder sourness. A variety of acetates such as sodium acetate can also be used.

The term “acetic acid concentration” used herein refers to a concentration that is represented in terms of acetic acid comprising acetic acid molecules (CH₃COOH) that are not dissociated, acetate ions (CH₃COO⁻) that are dissociated, and acetate that is not dissociated, for the following reasons. Whether the orally ingested acetic acid is an aqueous solution of acetic acid with a low pH level, neutralized acetate (for example, sodium acetate), or a dissociated acetate ion, the pH levels in the stomach or the intestinal canal after the small intestine where these substances are absorbed are not substantially affected by the composition, and are maintained at a substantially constant level in each site. Accordingly, the condition of an acetic acid molecule in the composition when it is put into the mouth does not affect the absorption of acetic acid in the body. Therefore, the composition of the present invention needs to comprise at least one member selected from among acetic acid, acetate ion, and acetate (hereafter it may be occasionally referred to as “acetic acids”).

Acetic acids can be assayed using, for example, a carboxylic acid analyzer (EYELA S-3000, Tokyo Rikakikai Co., Ltd.). This apparatus separates a variety of organic acids using columns and detects organic acids based on the principle whereby a reagent specifically reacts with a carboxyl group of organic acid. The use of this apparatus enables the quantification of acetic acids contained in the solution regardless of the dissociated or non-dissociated state thereof.

The composition of the present invention can be obtained by mixing at least one member selected from among acetic acid, acetate ion, and acetate with adequate amounts of other starting materials (starting materials for food or pharmaceuticals).

The form of the composition of the present invention is not particularly limited. Examples thereof include specified health foods (health foods), vinegared foods, sushi, marinades, beverages, and pharmaceuticals (tablets, capsules, powders, granules, fine grains, and drinkable preparations). The method for adding acetic acids is not particularly limited, and it may be carried out via a common technique. When acetic acid is used in the form of a solution with a low pH level, such as synthetic acetic acid or fermented vinegar, instead of the salt form, attention should be paid to “sourness” caused by lowered pH level from the viewpoint of ease of drinking or eating. More specifically, ingestion of a solution with a low pH level generates a discomforting feeling in the throat when the beverage slides down the throat, and the person gets the beverage stuck in the throat. When highly concentrated acetic acid is intended, for example, utilization of salt of acetic acid and/or encapsulation can be implemented.

The concentration of acetic acids to be added to the composition must be at least 6 mM in the case of a liquid. When a solid matter is included, the composition must comprise at least 0.36 g of acetic acids per 1,000 g of composition. If the concentration is lower than this level, ingestion of very large amounts of foods or beverages becomes necessary. Such concentration is calculated and determined based on the presumption that the daily intake of the composition is about 1,150 g in the case of foods and about 1 liter in the case of beverages.

About 0.5 g to 5 g of acetic acid (molecules) must be ingested per day. The necessary amount of active acetic acid molecules can be ingested by eating one serving or more per day in the case of vinegared food or sushi. Also, a necessary amount of active acetic acid molecules can be ingested by drinking about 50 ml to 1 liter of a beverage containing vinegar, such as cider vinegar, per day. Although ingestion of 5 g or more acetic acid (molecules) is possible, ingestion thereof in an amount exceeding 5 g is not preferable from the viewpoints of a taste of food, ease of eating, or ease of drinking.

When highly concentrated and unneutralized acetic acid is used, ingestion must be made with consideration for a disorder of the alimentary canal, such as stomach or intestine. According to the literature (Japan J. Pharmacol., vol. 41, p. 101, 1986; Med. Sci. Monit., vol. 5, p. 1031, 1999), the acetic acid concentration up to 3% exhibits the effect of protecting the gastric mucosa rather than damaging the stomach. Thus, direct ingestion of acetic acid at a concentration of approximately 3% in the form of a beverage will not cause any serious problems. There is an academic report concerning the LD₅₀ of acetic acid which was made using mice (the Journal of the Japanese Society of Nutrition and Food Science, vol. 36, p. 283, 1983). Based on those research results, ingestion of 5 ml of vinegar per kg of the body weight, i.e., ingestion up to 300 ml of vinegar at a time for an adult who weighs 60 kg, is not considered to cause a disorder of the alimentary canal.

Specifically, acetic acid can exhibit the effect of lowering blood pressure via the long-term oral ingestion thereof in amounts of approximately 0.5 g to 5 g per day. The term “long-term oral ingestion” refers to continual ingestion for at least 3 weeks. This is because the blood pressure was verified at a statistically significantly lower value in comparison with the control 3 weeks after the meal was changed to the test meal, based on the animal experiment.

The composition of the present invention is effective for preventing hypertension, particularly essential hypertension, the cause of which has not yet been clarified but which accounts for 90% of hypertension, cerebrovascular disorder, heart diseases, and other vascular lesions developed thereby.

### BRIEF DESCRIPTION OF THE DRAWINGS

FIG. 1 shows the acetic acid concentration in the hepatic portal vein of the control group and that of the group to which acetic acid had been administered (10 minutes, 30 minutes, and 60 minutes after ingestion).

FIG. 2 shows the acetic acid concentration in the abdominal artery of the control group and that of the group to which acetic acid had been administered (10 minutes, 30 minutes, and 60 minutes after ingestion).

FIG. 3 shows the acetic acid concentration in the large vein of the control group and that of the group to which acetic acid had been administered (10 minutes, 30 minutes, and 60 minutes after ingestion).

FIG. 4 shows change in the body weight of the control group and that of the group to which acetic acid had been administered.

FIG. 5 shows change in the feed consumption of the control group and that of the group to which acetic acid had been administered.

FIG. 6 shows change in the water intake of the control group and that of the group to which acetic acid had been administered.

FIG. 7 shows change in the blood pressure level of the control group and that of the group to which acetic acid had been administered.

FIG. 8 shows change in the heart rate of the control group and that of the group to which acetic acid had been administered.

FIG. 9 shows change in the blood pressure level of the control group and that of the group to which acetic acid had been administered (addition of 0.36%, 0.72%, 1.5%, 3%, 6%, and 9% acetic acid).

This description includes part or all of the contents as disclosed in the descriptions of Japanese Patent Application Nos. 2000-394632 and 2001-298211, which are priority documents of the present application.

### Preferred Embodiments of the Invention

The present invention is hereafter described with reference to the following examples and reference example, although the technical scope of the present invention is not limited thereto.

### REFERENCE EXAMPLE

### Test for Oral Ingestion of Acetic Acid Using Pig

1. Method

(1) Test Material

As test materials, 150 ml of distilled water or 150 ml of a 6% (w/w) aqueous solution prepared by diluting a reagent acetic acid (a special grade) with distilled water were prepared and then employed as samples.

(2) Method of Administration

As test animals, 5 pigs (with body weights of about 20 kg) were prepared for each group to which each sample was to be administered. These test animals were subjected to fasting from the night before testing. A tranquilizer was administered on the day of testing. After the test animals were confirmed to be under sedation, samples were administered orally to the stomach.

(3) Sacrifice After Administration

The test animals were sacrificed 10 minutes after the administration of water, and 10 minutes, 30 minutes, and 60 minutes after the administration of an aqueous solution of acetic acid.

(4) Items to be Assayed

Blood was sampled from the hepatic portal vein, the abdominal artery, and the postcaval vein, and the acetic acid concentrations in the serums were assayed by gas chromatography.

2. Results

The acetic acid concentrations in serums were as shown below.

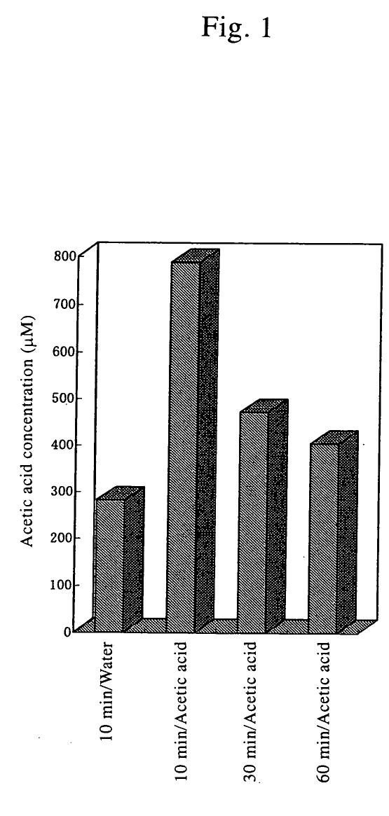

(1) Hepatic portal vein: as shown in FIG. 1. The acetic acid concentration of the group assayed 10 minutes after ingestion was significantly higher than that of other 3 groups (significant difference was observed via ANOVA: p<0.05).

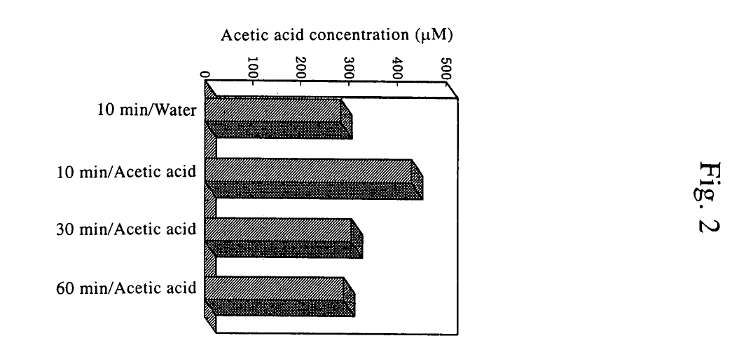

(2) Abdominal artery: as shown in FIG. 2. The acetic acid concentration of the group assayed 10 minutes after ingestion was significantly higher than that of other 3 groups (significant difference was observed via ANOVA: p<0.05).

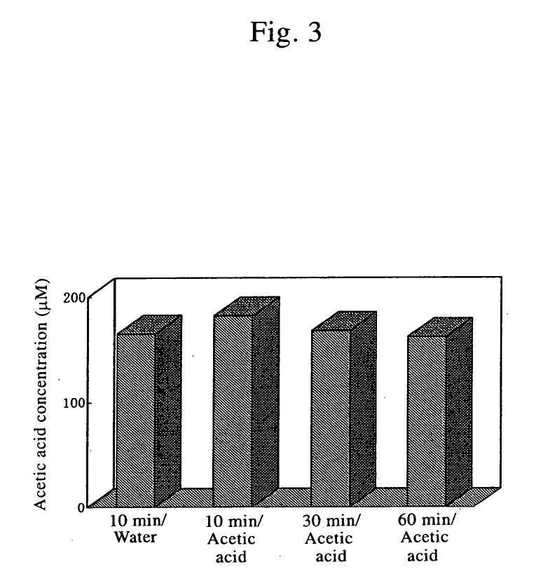

(3) Postcaval vein: as shown in FIG. 3. There was no significant difference among groups (p>0.05).

Although the acetic acid concentration became relatively high in the portal vein 10 minutes after ingestion of an aqueous solution of about 100 mM acetic acid, it rapidly decreased with the elapse of time. As with the case in the portal vein, the maximal level was marked in the artery 10 minutes after ingestion, although it returned to the ordinary level 30 minutes later. Since acetic acid is rapidly absorbed and utilized in the periphery, the acetic acid concentration in the large vein was not substantially elevated in spite of acetic acid ingestion. The maximal acetic acid concentration was marked in the portal vein 10 minutes after acetic acid ingestion, and the concentration at that time was about 0.8 mM. This indicates that the acetic acid concentration is diluted to 1/125 of that at the time of administration thereof.

Thus, the rate of acetic acid metabolism was found to be rapid, and the concentration thereof was found to return to a level substantially the same as that under fasting conditions 30 minutes after ingestion.

Human clinical study cannot be conducted due to the necessity of sacrificing subjects. Based on the fact that the body weight of a pig is about 20 kg, the obtained results are converted to those in terms of a human adult (60 kg). This would be equivalent to a human adult drinking 450 ml of a beverage at one time.

It was judged that the acetic acid concentration in blood cannot be elevated to the concentration thereof in a dialysate used for a dialysis treatment for humans (8 to 35 mM) via ingestion of acetic acid in the form of beverage.

### EXAMPLE 1

### Test for Confirming the Effect of Acetic Acid Using SHR

1. Method

(1) Test Substance

Acetic acid (reagent, special grade) was diluted with distilled water to prepare a 5% (w/v) aqueous solution, and this aqueous solution was added to powdery feeds in an amount of 3% to prepare feeds (a test meal). Also, powdery feed (a control) to which no substance had been added was prepared. These feeds were employed as samples.

(2) Method of Administration

As test animals, 6 spontaneous hypertensive rats (SPF, SHR/NCrj, male, 4-week-old, Charles River Japan) were prepared for each group to which each sample was to be administered (the group to which the test meal was to be administered and the control group). Rats were allowed to freely ingest feeds and tap water.

(3) Period of Administration

The day on which the test meal had been first administered was determined to be the day 1, and administration was continued for 8 weeks.

(4) Test System

The test animals were kept under the following conditions for 5 days before the test.

They were kept at a temperature of 20° C. to 26° C. and a humidity of 40% to 70% and placed in a well-lit place for 12 hours a day and in a dark place for 12 hours a day.

(5) Measurement of Blood Pressure and Heart Rate

The blood pressure and the heart rate were noninvasively measured every week. The blood pressure was measured using an apparatus for noninvasive blood pressure measurement by the tail-cuff method. The heart rate was measured by employing the pulse of the blood pressure as a trigger. Measurements were carried out 5 times, and the mean thereof was determined.

2. Results

(1) Increase in Body Weight, Feed Consumption, and Water Consumption

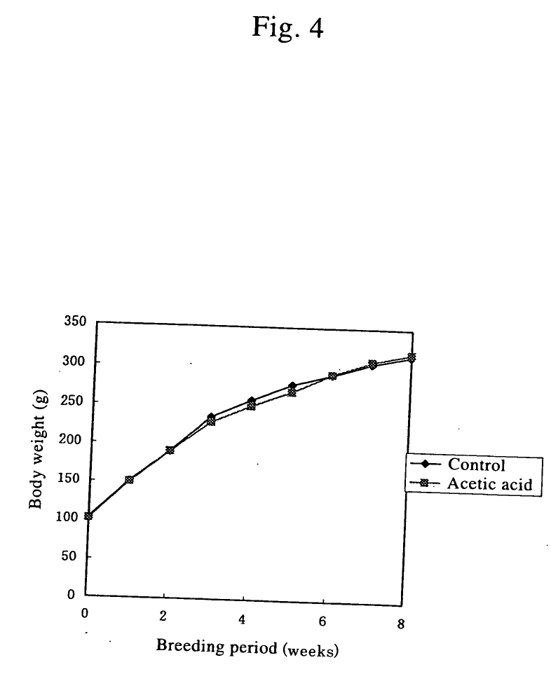

When the test was initiated, the average body weight of the control group was 102.5 g, and that of the test group was 101.8 g. That is, there was no statistically significant difference. As shown in FIG. 4, there was no significant difference between the body weight of individuals in the control group and that of the group to which the test meal had been administered 8 weeks later (p>0.05 according to the t-test).

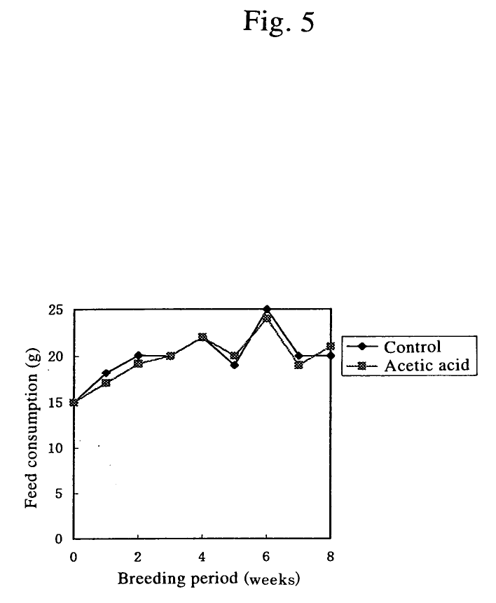

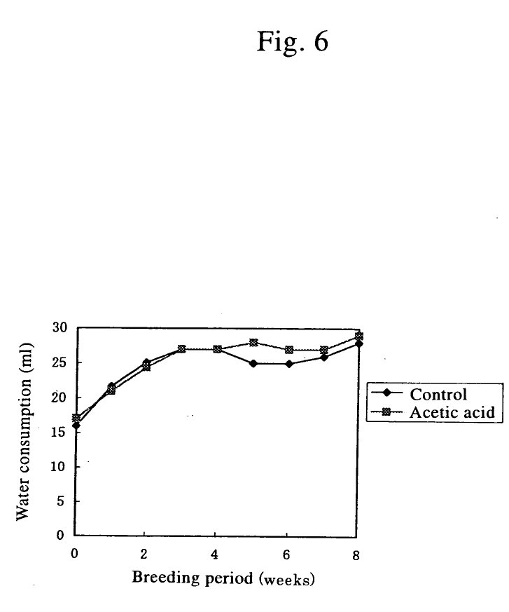

Also, there was no significant difference in feed consumption (FIG. 5) or water consumption (FIG. 6) between two groups (t-test; the p<0.05 level was determined to be significantly different).

(2) Blood Pressure and Heart Rate

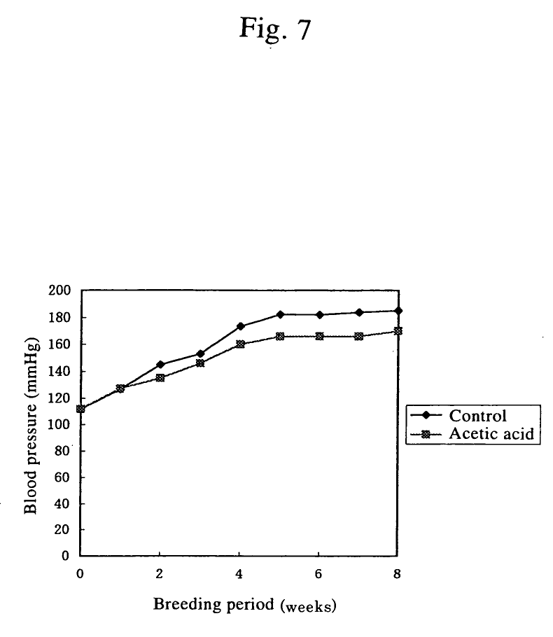

The blood pressure level of the group to which the test meal had been administered became significantly lower than that of the control group from the 4th week to the 8th week. Thus, the elevation of blood pressure was found to be inhibited (FIG. 7) (p<0.05).

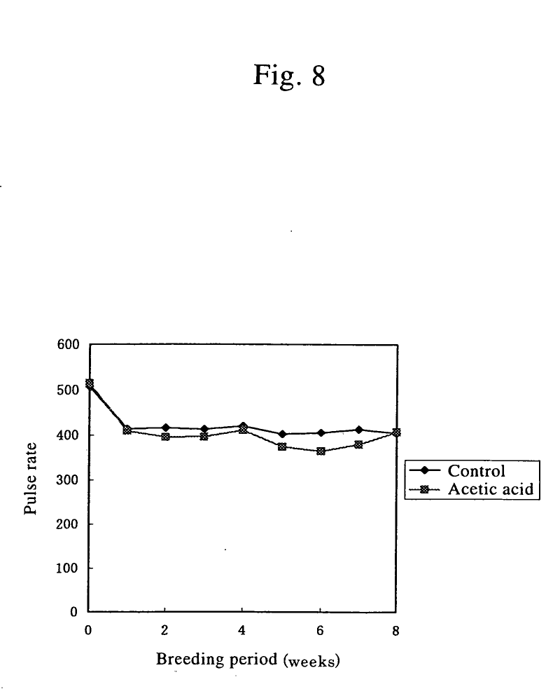

The heart rate varied in a substantially similar manner as with the case of the blood pressure (FIG. 8), although the heart rate did not become significantly different from that of the control group at any stage.

### EXAMPLE 2

### Culturing Test of SHR Using Feed With Varied Doses of Acetic Acid

The experimental method and conditions were substantially in accordance with Example 1 except for the lineage of SHR, the kind of feed, and the amount of acetic acid added to the feed.

Acetic acid (reagent, special grade) was diluted with distilled water to prepare a 5% (w/v) aqueous solution. This aqueous solution was added to powdery feeds (Labo MR Stock, Nousan Corporation, requiring sterilization before use) in amounts of 0.36%, 0.72%, 1.5%, 3%, 6%, and 9%, respectively. The resulting feeds having different acetic acid concentrations were to be independently administered to the test groups. Powdery feeds containing no additives were to be administered to another group (the control group). Thus, seven experimental plots were prepared in total.

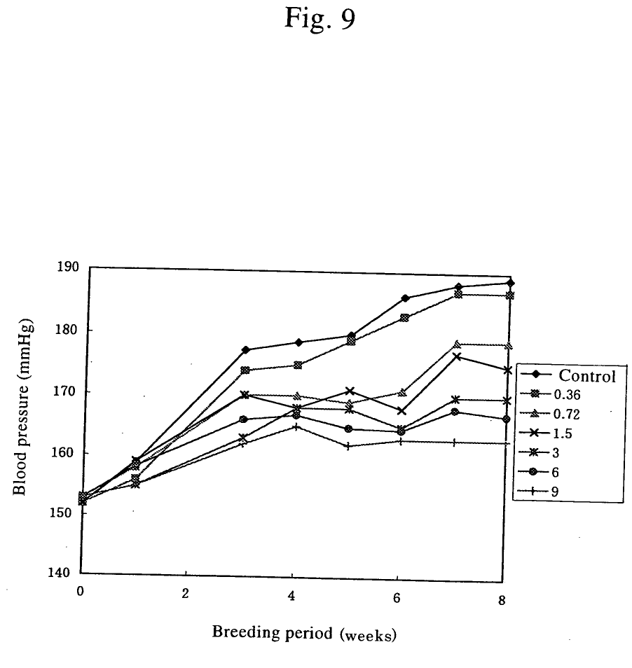

Animals used were the 4-week-old SPF SHR/Hos rats, and each group consisted of 6 individuals. The blood pressure was continually measured until the 8th week (FIG. 9), and the blood pressure level of the group to which the feeds comprising 0.36% acetic acid had been administered was not significantly different from that of the control group at any stage. The blood pressure of the group to which the feeds comprising 0.72% acetic acid had been administered became significantly lower than that of the control group on the 5th week, the blood pressure of the groups to which the feeds comprising 1.5% acetic acid had been administered became significantly lower than that of the control group on the 4th week, and the blood pressure of the groups to which the feeds comprising 3% to 9% acetic acid had been administered became significantly lower than that of the control group on the 3rd week (significant difference was observed via ANOVA in all these cases: p<0.05). This demonstrates that the feeds must comprise 0.72% or more of a 5% acetic acid solution, i.e., 0.36 g or more acetic acid (molecules) per 1,000 g of feeds, in order to inhibit the elevation of blood pressure. There was no significant difference among groups in terms of the body weights and the feed consumption. (ANOVA: the body weight of the control group was 71.8 g on the week 0 and 275.7 g on the 8th week; and the feed consumption of the control group was 15.1 g on the week 0 and 20.5 g on the 7th week.)

The blood pressure levels of the test groups became significantly lower than that of the control group3 weeks after rats began to eat the acetic acid-containing feeds. There was no significant difference until the 2nd week. This indicates that acetic acid must be continually ingested for at least 3 weeks and that substantially no effect is attained by ingestion thereof for less than 2 weeks.

According to the most recent Japan's national survey (Kokumin eiyou no genjo (Current Status of National Nutrition), Kenko Eiyo Joho Kenkyukai (Society for Health and Nutrition Studies) (ed.), Dai-ichi Shuppan Publishing, Co., Ltd., p. 78, 2000), a Japanese person ingests 1,116 g of food on average per day except for flavor enhancers, nonessential beverages, and beverages such as milk or fruit juice. Accordingly, if the content with which the aforementioned significant difference was attained (0.72% to 9% of an aqueous solution of 5% acetic acid contained in the feeds) is converted in relation to 1,116 g, the amount of acetic acid (molecules) is about 0.5 g to 5 g. Specifically, prevention of hypertension can be expected from the ingestion of acetic acid (molecules) in amounts of 0.5 g to 5 g on average per day.

### EXAMPLE 3

### Production Example of Composition (Beverage) and Evaluation Thereof

A beverage having the following composition was produced. Specifically, 5 g of acetic acid (food additive) and 0.2 g of sucralose (food additive) were added to water, and these substances were mixed to prepare 1 liter of a solution. Thus, a beverage was obtained. The concentration of acetic acid molecules in this beverage was 83 mM.

This beverage has excellent drinkability with moderate sourness and a refreshing taste. Daily ingestion thereof in amounts of about 100 ml to 1 liter (0.5 g to 5 g of acetic acid molecules) for a long period of time (3 weeks or longer) is considered to be able to prevent hypertension by the effects attained by acetic acid molecules.

Also, 20 adults participated in a tasting test. The acetic acid content in 1,000 g of the beverage was set at 5 different levels: 1 g; 5 g; 10 g; 30 g; and 50 g. The resulting beverages were then evaluated, and as a result, 19 participants pointed out a problem of taste when acetic acid content was 50 g. Only 10 participants recognized a problem -of taste when acetic acid content was 30 g, and 18 or more participants indicated ease of drinking when acetic acid content was 10 g or lower. Thus, the preferable acetic acid content in the beverage was determined to be 30 g or lower per 1,000 g of the beverage.

### EXAMPLE 4

### Production Example of Food

Acetic-acid-containing food (4 servings) having the following composition was produced. Specifically, 12 sticks of green asparagus, 2 pieces of bacon, 2 g of dried bonito flakes, 30 ml of soy sauce, 2.3 g of acetic acid (food additive), and 45 ml of tap water were thoroughly mixed with each other to prepare acetic-acid-containing food. The concentration of acetic acid molecules in the liquid portion of this food was about 500 mM (containing about 30 g of acetic acid molecules per liter of liquid).

This food is a vinegared food having excellent edibility with moderate sourness and a refreshing taste. Daily ingestion thereof in amounts of one serving per day to one serving per meal, i.e., about three servings per day (0.58 g to 1.73 g of acetic acid molecules per day) for a long period of time is considered to inhibit the elevation of blood pressure by the effects attained by acetic acid molecules, thereby preventing hypertension.

### EXAMPLE 5

### Production Example of Food

Acetic-acid-containing food having the following composition was produced in the following manner. Specifically, 3 gou (translator's note: 1 gou=180 cc) of rice, 3 gou of water, 3 pieces of lightly-salted salmon, 4 pieces of shiitake mushrooms (long thin strips), a package of shimeji mushrooms, thinly-sliced fried egg prepared from 3 eggs, 10 leaves of green perilla (long thin strips), a mixed seasoning A (a mixture of 2 tablespoonfuls of sake (Japanese rice wine), 2 tablespoonfuls of water, and a pinch of salt), and a mixed seasoning B (a mixture of 4 tablespoonfuls of rice vinegar (acetic acid concentration of 4.5%), 5 tablespoonfuls of sugar, and 2 teaspoonfuls of salt) were first prepared as ingredients.

(1) Roasted lightly-salted salmon was put on rice and rice was cooked in that state, followed by steaming. (2) The salmon was taken out, bones and skin were removed, and the fish meat was broken up into large pieces. (3) Shiitake mushrooms and shimeji mushrooms were combined and steam-steeped in the seasoning A. (4) The cooked rice was dressed with the seasoning B to prepare sushi rice (rice seasoned with vinegar). (5) The flaked salmon and the steam-steeped shiitake and shimeji mushrooms were added thereto and mixed. The thinly-sliced fried egg and the green perilla (long thin strips) were sprinkled thereon.

This food is delicious sushi with moderate sourness and comprises about 2.7 g of acetic acid molecules. By eating about 1/4 to 1/3 of the finished food (0.68 g to 0.9 g of acetic acid molecules) one to three times a day (0.68 g to 2.7 g of acetic acid molecules in total), the elevation of blood pressure is inhibited, and hypertension can thereby be prevented.

### EXAMPLE 6

### Production Example of Beverage

A beverage having the following composition was produced. Specifically, 5 g of acetic acid (food additive) and 0.5 g of stevioside (food additive) were added to water, and these substances were mixed to prepare 1 liter of a solution. Thus, a beverage was produced. The concentration of acetic acid molecules in this beverage was 83 mM.

This beverage has excellent drinkability with moderate sourness and a refreshing taste. Daily ingestion thereof in amounts of about 100 ml to 1 liter (0.5 g to 5 g of acetic acid molecules) for a long period of time is considered to inhibit the elevation of blood pressure by the effects attained by acetic acid molecules, thereby preventing hypertension.

### EXAMPLE 7

### Production Example of Beverage

A beverage having the following composition was produced. Specifically, 2 teaspoonfuls of cider vinegar (acetic acid concentration of 5%), 2 teaspoonfuls of honey, and 150 ml of chilled water were mixed with each other to prepare a solution. Thus, a beverage was produced. The concentration of acetic acid molecules in this beverage was about 120 mM.

This beverage has excellent drinkability with moderate sourness and a refreshing taste. Daily ingestion thereof in amounts of about 100 ml to 700 ml (0.7 g to 5 g of acetic acid molecules) for a long period of time is considered to inhibit the elevation of blood pressure by the effects attained by acetic acid molecules, thereby preventing hypertension.

### EXAMPLE 8

### Production Example of Powder

Acetic acid was allowed to adsorb onto dextrin and then dehydrated. Thus, powders comprising 15% (w/w) of acetic acid were prepared. These powders (6% (w/w)) were added to powders comprising sugar, skimmed milk powder, and lactose (94% (w/w)), and they were thoroughly mixed with each other to prepare powders.

Such powders have moderate sourness. Oral ingestion thereof in amounts of 100 g per day (0.9 g of acetic acid molecules) for a long period of time is considered to inhibit the elevation of blood pressure, thereby preventing hypertension.

### EXAMPLE 9

### Production Example of Tablet

Tablets comprising 1.25 g (25 mg per tablet) of sodium acetate per 100 g thereof were produced. Oral ingestion of these tablets in amounts of 70 g per day (35 tablets, 0.63 g of acetic acid molecules) for a long period of time is considered to inhibit the elevation of blood pressure, thereby preventing hypertension.

### EXAMPLE 10

### Test for Confirming the Effect of Acetic Acid on Hypertensive Patient

1. Method

(1) Test Beverage

As test beverages, a beverage containing 1.5 g of acetic acid, a beverage containing 0.75 g of acetic acid and 1 g of lactic acid, and a placebo beverage containing 2 g of lactic acid instead of acetic acid were used (100 ml/bottle). The composition of each test beverage is shown in Table 1.

These 3 beverages had substantially the same appearances, sweet tastes, salty tastes, and the like, although there were some variations in terms of sourness. It was confirmed that they couldn't be distinguished from each other based on their appearance. As acetic acid, vinegar having moderate sourness and a preferable flavor (cider vinegar, Mizkan, acetic acid concentration of 5% (w/v)) was used. Fermented lactic acid was used.

TABLE 1
Test beverage composition

Vinegar-containing
beverage

| | High-dose | Low-dose | Placebo beverage |
|---|---|---|---|
| Acetic acid (mg) | 1500 | 750 | 0 |
| Lactic acid (mg) | 0 | 1000 | 2000 |
| Water (g) | 94.8 | 95.5 | 96.3 |
| Protein (g) | 0 | 0 | 0 |
| Fat (g) | 0 | 0 | 0 |
| Carbohydrate (g) | 4.2 | 3.5 | 2.5 |
| Ash content (g) | 1 | 1 | 1 |
| Sodium (mg) | 5 | 5 | 5 |
| Potassium (mg) | 550 | 550 | 550 |
| Calcium (mg) | 60 | 60 | 60 |
| Vitamin C (mg) | 50 | 50 | 50 |
| Total (g) | 102 | 102 | 102 |
| Calories (kcal) | 15 | 12 | 8 |

(2) Subjects
1) Basis of Selection

Patients with mild to moderate hypertension who had continuously had systolic blood pressure of 140 to 180 mmHg or diastolic blood pressure of 90 to 105 mmHg for 3 months before the initiation of the test were subjected to the test. However, those having secondary hypertension, those diagnosed by a doctor as being in immediate need of an antihypertensive agent administration, those having alcohol dependence or severe anemia, and those afflicted with serious diseases were excluded. Also, those who had routinely taken some kind of medicine for internal application and those who had routinely taken supplements that might affect blood pressure were excluded.

2) Subjects

Volunteers (57 individuals) who met the aforementioned requirements were divided into 3 groups independently consisting of 19 individuals so as to have matching conditions in terms of systolic blood pressure, diastolic blood pressure, age, and sex among groups. Among those 57 individuals, a total of 6 individuals, i.e., 2 individuals who had failed to drink the test beverage for 8 days or longer during the ingestion period for personal reasons and 4 individuals who could not visit the hospital on the testing day, were eliminated as subjects of data analysis.

As a result, the number of available subjects for the analysis was 51. They were 31 males and 20 females aged 51.2±8.1. There were no significant differences among the 3 groups in terms of age, sex, body weight, BMI, blood pressure, or pulse.

(3) Outline of Test

1) Testing Method

The double blind method was adopted. A group to which an acetic-acid-containing beverage was to be administered, a group to which another acetic-acid-containing beverage having a different acetic acid content was to be administered, and a group to which a placebo beverage was to be administered were subjected to parallel comparison.

2) Testing Period

The testing period was a total of 15 weeks consisting of 3 weeks of a pre-observation period (a non-ingestion period), 8 weeks of an ingestion period, and 4 weeks of a post-observation period (a non-ingestion period).

3) Method of Ingestion

Test beverages were ingested by drinking a bottle of 100 ml of the placebo beverage, 100 ml of the acetic-acid-containing beverage containing 0.75 g of acetic acid, or 100 ml of the acetic-acid-containing beverage containing 1.5 g of acetic acid once a day in the morning while maintaining customary eating habits. The subjects were instructed to refrain from immoderate eating and drinking and not to change other routines during the test period.

(4) Items and Method of Testing

1) Blood Testing and Urine Testing

Besides the preliminary test conducted before the initiation of the test, blood testing and urine testing were carried out two times, i.e., at the end of the pre-observation period (immediately before the ingestion period) and at the end of the ingestion period during the test period.

Blood sampling was carried out from 8:30 am to 11:00 am under fasting conditions by refraining from eating and drinking after 9:00 pm on the previous night. The blood testing was requested from a company that conducts clinical examinations. In order to avoid extrinsic influences on the serum protein level, the serum lipid level, and the renin activity, blood sampling was carried out in a sitting position after the subject was allowed to rest for 10 minutes or longer.

Urine testing was carried out via a paper test, and glucose, protein, urobilinogen, and occult blood were examined.

2) Blood Pressure and Physical Measurement

The blood pressure and the pulse were measured between 8:30 am and 11:30 am, and every subject was assigned a fixed time for measurement. The subjects were to visit the hospital without ingesting anything except for water and the test beverage on the testing day, allowed to rest for 10 minutes or longer after the visit, and subjected to measurement three times at intervals of 1 minute at the elbow of the left arm in a sitting position while being dressed but without wearing shoes. The blood pressure and the frequency of pulse marked when the median of the systolic blood pressure had been obtained were determined as the data for that day. Blood pressure was measured 8 times in total during the test: before the pre-observation period; immediately before the ingestion period; the 2nd, 4th, 6th, and 8th weeks after the initiation of the ingestion; and the 2nd and 4th weeks after the completion of the ingestion (the post-observation period).

The body weight was measured at the time of diagnosis and interview by a doctor. When measuring the body weight, it was attempted to avoid the influence caused by clothing as great an extent as possible.

3) Diagnosis and Interview

Diagnosis and interview were conducted by a doctor, and all adverse events were recorded. In particular, development of subjective symptoms and that of side effects were explored in detail.

(5) Method of Analysis

The comparison of the blood pressure and the heart rate among groups during the ingestion period was conducted via two-way analysis of variance (ANOVA). The primary effects and the interaction between the test beverage ingested and the period of ingestion were analyzed. Further, when the primary effects were attained during the ingestion period, the measured blood pressure level was compared with the blood pressure level immediately before the ingestion via Dunnett's multiple comparison. Other measured values and the results of blood testing of the patients were subjected to a paired t-test, and interaction with the ingested test beverage was tested by t-test. The SPSS Ver. 10 statistical software (SPSS) was used, and the level of significance was determined to be 5% or lower for both tests.

2. Results

(1) Blood Pressure and Heart Rate

Changes in blood pressure levels and heart rates during the test are shown in Table 2. The systolic blood pressure levels were analyzed with a two-way analysis of variance. As a result, changes in the systolic blood pressure levels were different among 3 groups during the ingestion period (p<0.05).

The blood pressure level in the group that had ingested 1.5 g of acetic acid per day (hereafter this group is referred to as the “high-dose group”) and that in the group that had ingested 0.75 g of acetic acid per day (hereafter this group is referred to as the “low-dose group”) began to become significantly lower from the 6th week after the initiation of ingestion to the end of the ingestion period. In contrast, no significant change occurred in the blood pressure level of the group that had ingested the placebo beverage (hereafter this group is referred to as the “placebo group”) throughout the ingestion period.

Based on the blood pressure level immediately before ingestion, changes in the systolic blood pressure level of the high-dose group became significantly different from those of the placebo group after continual ingestion for 4 weeks. Also, such significant difference was observed after continual ingestion for 6 weeks in the case of the low-dose group (Table 3). There was no significant difference between the high-dose group and the low-dose group.

As a result of the two-way analysis of variance for the systolic blood pressure level, there was no interaction found between the test beverage and the ingestion period, and the primary effect was observed during the ingestion period (p<0.05). The blood pressure level of the high-dose group began to become significantly lower from 4th week after the initiation of continual ingestion and that of the low-dose group began to become significantly lower from 6th week after the initiation of continual ingestion (p<0.05). In contrast, no significant change occurred in the placebo group during the ingestion period.

Based on the blood pressure level immediately before ingestion, changes in the systolic blood pressure level of the high-dose group became significantly different from those of the placebo group after continual ingestion for 4 weeks.

TABLE 2
Changes in blood pressure levels and heart rates

| | | 3 weeks before | Baseline (immediately | Period of beverage ingestion | |
|---|---|---|---|---|---|
| | | ingestion | before ingestion) | 2nd week | 4th week |
| SBP | High-dose | 152.1 ± 8.4 | 151.7 ± 13.2 | 147.2 ± 14.6 | 141.1 ± 17.3* |
| (mmHg) | Low-dose | 151.7 ± 7.5 | 151.8 ± 8.6 | 151.8 ± 10.9 | 146.1 ± 19.0 |
| | Placebo | 151.6 ± 7.0 | 151.1 ± 9.1 | 150.5 ± 13.6 | 151.8 ± 13.9 |
| DBP | High-dose | 90.8 ± 7.8 | 89.5 ± 6.1 | 88.0 ± 7.8 | 84.5 ± 7.4** |
| (mmHg) | Low-dose | 90.9 ± 10.8 | 89.9 ± 9.6 | 87.2 ± 11.3 | 86.1 ± 11.4 |
| | Placebo | 89.8 ± 7.0 | 90.1 ± 6.0 | 89.9 ± 8.4 | 90.4 ± 7.7 |
| heart rate | High-dose | 70.5 ± 7.8 | 69.1 ± 7.5 | 70.7 ± 10.3 | 67.7 ± 10.1 |
| (beats/min) | Low-dose | 72.6 ± 5.3 | 71.4 ± 7.1 | 74.1 ± 9.4 | 71.4 ± 9.4 |
| | Placebo | 73.3 ± 5.8 | 71.4 ± 6.3 | 71.9 ± 6.5 | 73.4 ± 8.8 |

| | | Period of beverage ingestion | | 2 weeks after the | 4 weeks after the |
|---|---|---|---|---|---|
| | | 6th week | 8th week | end of ingestion | end of ingestion |
| SBP | High-dose | 136.7 ± 15.7*** | 136.9 ± 16.6** | 148.1 ± 16.5 | 147.9 ± 16.4 |
| (mmHg) | Low-dose | 141.7 ± 14.4** | 141.2 ± 16.9** | 148.1 ± 11.6 | 148.6 ± 14.2 |
| | Placebo | 151.8 ± 11.8 | 152.8 ± 8.5 | 151.0 ± 10.2 | 150.2 ± 12.3 |
| DBP | High-dose | 83.1 ± 7.5*** | 83.0 ± 7.1*** | 88.4 ± 6.2 | 88.4 ± 9.7 |
| (mmHg) | Low-dose | 84.8 ± 11.1* | 84.8 ± 10.1* | 88.5 ± 11.3 | 88.9 ± 9.3 |
| | Placebo | 89.6 ± 7.1 | 89.9 ± 7.5 | 89.4 ± 7.8 | 88.4 ± 8.7 |
| heart rate | High-dose | 68.6 ± 11.7 | 67.7 ± 10.1 | 68.3 ± 10.2 | 68.2 ± 8.5 |
| (beats/min) | Low-dose | 72.8 ± 10.5 | 70.7 ± 10.0 | 71.9 ± 7.4 | 69.6 ± 6.7 |
| | Placebo | 71.4 ± 8.1 | 72.2 ± 7.5 | 71.1 ± 9.1 | 71.3 ± 8.5 |

SBP: Systolic blood pressure
DBP: Diastolic blood pressure
*p < 0.05;
**p < 0.01;
***p < 0.001

TABLE 3
Changes in blood pressure levels and heart rates

| | | | | | | | 2 weeks after | 4 weeks after |
|---|---|---|---|---|---|---|---|---|
| | | Base- | Period of ingesting beverage | | | | the end of | the end of |
| | | line | 2nd week | 4th week | 6th week | 8th week | ingestion | ingestion |
| SBP | High- | 0.0 | −4.4 ± 11.5 | −10.6 ± 16.3 | −14.5 ± 14.1 | −14.8 ± 17.5 | −3.5 ± 12.1 | −3.8 ± 14.7 |
| (mmHg) | dose | | | p < 0.05 | p < 0.001 | p < 0.01 | | |
| | Low- | 0.0 | 0.0 ± 9.5 | −5.7 ± 16.7 | −10.1 ± 11.7 | −10.6 ± 14.8 | −3.7 ± 11.3 | −3.2 ± 12.5 |
| | dose | | | | p < 0.01 | p < 0.01 | | |
| | Placebo | 0.0 | −0.6 ± 10.9 | 0.7 ± 13.5 | 0.8 ± 11.0 | 1.7 ± 10.8 | −0.1 ± 14.9 | −0.9 ± 16.9 |
| DBP | High- | 0.0 | −1.5 ± 4.2 | −4.9 ± 6.0 | −6.4 ± 6.1 | −6.5 ± 6.6 | −1.1 ± 5.7 | −1.1 ± 6.9 |
| (mmHg) | dose | | | p < 0.05 | p < 0.05 | p < 0.01 | | |
| | Low- | 0.0 | −2.7 ± 8.1 | −3.8 ± 11.1 | −5.1 ± 9.6 | −5.1 ± 9.3 | −1.4 ± 8.4 | −1.0 ± 7.4 |
| | dose | | | | | | | |
| | Placebo | 0.0 | −0.2 ± 6.8 | 0.3 ± 6.5 | −0.5 ± 7.2 | −0.2 ± 3.7 | −0.7 ± 8.4 | −1.7 ± 6.5 |
| heart rate | High- | 0.0 | 1.6 ± 5.1 | −1.4 ± 4.6 | −0.5 ± 6.5 | −1.4 ± 5.3 | −0.8 ± 4.4 | −0.9 ± 5.2 |
| (beats/min) | dose | | | | | | | |
| | Low- | 0.0 | 2.7 ± 3.9 | 0.1 ± 5.4 | 1.4 ± 7.6 | −0.7 ± 6.0 | 0.5 ± 5.1 | −1.8 ± 5.1 |
| | dose | | | | | | | |
| | Placebo | 0.0 | 0.5 ± 4.7 | 2.0 ± 5.9 | 0.0 ± 6.9 | 0.8 ± 6.3 | −0.3 ± 4.8 | −0.1 ± 4.3 |

SBP: Systolic blood pressure
DBP: Diastolic blood pressure

(2) Body Weight and BMI

BMI did not significantly change between the periods before and after ingestion in either group.

(3) Blood Testing and Urine Testing

Although there were some significant differences concerning some parameters between the periods before and after ingestion in all groups, these slight changes were within the normal ranges. As a result of investigation regarding each case, the measured value did not vary from the normal range to an abnormal range in any case.

According to the urine testing, examples of occult hematuria or unusual change were observed in all groups, although this abnormality wasn't problematic.

(4) Diagnosis and Interview

No difference was observed in the rate of adverse events occurring among the groups that had ingested the test beverages. Also, the causal relationship between the test beverage and any of the adverse events was judged to be poor. According to the diagnosis and interview, no case was observed in any group that should be especially noted.

3. Conclusions

Accordingly, continual ingestion of about 0.75 g or 1.5 g of acetic acid per day for at least 4 weeks was found to significantly lower the blood pressure level of a patient with mild to moderate hypertension. Also, the composition of the present invention was found to be effective for such hypertensive patient. Further, an acetic acid-containing beverage was verified to pose no problems in terms of safety.

All publications, patents, and patent applications cited herein are incorporated herein by reference in their entirety.

### INDUSTRIAL APPLICABILITY

The present invention provides a composition that can inhibit the elevation of blood pressure and can be safely used with a sense of security. The composition according to the present invention that can inhibit the elevation of blood pressure is particularly effective for inhibiting the elevation of blood pressure of people who have borderline blood pressure or who are fated to develop essential hypertension.

1. Food or a pharmaceutical composition for preventing hypertension, which inhibits the elevation of blood pressure via the long-term oral ingestion thereof and comprises at least one member selected from among acetic acid, acetate ion, and acetate.

2. The composition according to claim 1, which comprises at least one member selected from among acetic acid, acetate ion, and acetate in amounts of 0.36 g to 30 g in total (in terms of acetic acid) per 1,000 g of composition.

3. The composition according to claim 1, wherein the intake of at least one member selected from among acetic acid, acetate ion, and acetate is adjusted to 0.5 g to 5 g in total (in terms of acetic acid) per day.

4. The composition according to claim 1, wherein the period of ingestion is 3 weeks or longer.

5. A method for preventing, ameliorating, or treating hypertension which comprises a long-term oral ingestion of food or a pharmaceutical composition comprising at least one member selected from among acetic acid, acetate ion, and acetate.

6. The method according to claim 5, wherein the elevation of blood pressure is inhibited or reduced via the long-term oral ingestion of the composition.

7. The method according to claim 5, wherein the composition comprises at least one member selected from among acetic acid, acetate ion, and acetate in amounts of 0.36 g to 30 g in total (in terms of acetic acid) per 1,000 g of composition.

8. The method according to claim 5, wherein at least one member selected from among acetic acid, acetate ion, and acetate is ingested in amounts of 0.5 g to 5 g in total (in terms of acetic acid) per day.

9. The method according to claim 5, wherein the period of ingestion is 3 weeks or longer.

10. A method for inhibiting or reducing the elevation of blood pressure which comprises a long-term oral ingestion of food or a pharmaceutical composition comprising at least one member selected from among acetic acid, acetate ion, and acetate.

11. The method according to claim 10, wherein the composition comprises at least one member selected from among acetic acid, acetate ion, and acetate in amounts of 0.36 g to 30 g in total (in terms of acetic acid) per 1,000 g of composition.

12. The method according to claim 10, wherein at least one member selected from among acetic acid, acetate ion, and acetate is ingested in amounts of 0.5 g to 5 g in total (in terms of acetic acid) per day.

13. The method according to claim 10, wherein the period of ingestion is 3 weeks or longer.
